(19) United States
(12) Patent Application Publication
(10) Pub. No.: US 2023/0030766 A1
(43) Pub. Date: Feb. 2, 2023

(54) SYSTEMS AND METHODS FOR CALIBRATING OXYGEN SENSORS IN VENTILATORS

(71) Applicant: Covidien LP, Mansfield, MA (US)

(72) Inventor: Julio JENARO, Galway (IE)

(73) Assignee: Covidien LP, Mansfield, MA (US)

(21) Appl. No.: 17/870,023

(22) Filed: Jul. 21, 2022

Related U.S. Application Data
(60) Provisional application No. 63/228,460, filed on Aug. 2, 2021.

(51) Int. Cl. G01N 33/00; A61M 16/00; A61M 16/10

(52) U.S. Cl. CPC G01N 33/0006; A61M 16/0003; A61M 16/1005; A61M 2016/1025

(57) ABSTRACT

Systems and methods for calibrating oxygen sensors in ventilators are provided. An oxygen sensor is coupled in flow communication with a first oxygen gas source. A calibration circuit including a second oxygen gas source is coupled in flow communication with the oxygen sensor and a third oxygen gas source is coupled in flow communication with the oxygen sensor. A controller is configured to determine a calibration curve for the oxygen sensor via the calibration circuit by measuring the second oxygen gas source and the third oxygen gas source. Based on the calibration curve, an oxygen concentration value of the first oxygen gas source is measured and distributed.

### CROSS-REFERENCE TO RELATED APPLICATIONS

This application claims the benefit of U.S. Provisional Application No. 63/228,460 filed 2 Aug. 2022, titled “Systems and Methods for Calibrating Oxygen Sensors in Ventilators,” which is incorporated herein by reference in its entirety.

### INTRODUCTION

Medical ventilator systems are used to provide ventilatory and supplemental oxygen support to patients. These ventilators typically are connected to an oxygen gas flow for operation. Additionally, the ventilators include an oxygen sensor that is used for controlling the oxygen support to the patients. This oxygen sensor, however, can require constant periodic calibration because of sensor drift over time. Additionally, oxygen gas flow can include a wide range of concentration levels (e.g., 90%-100%), and the oxygen concentration level of the gas flow is desired to be known by the ventilator for calibration of the oxygen sensor.

It is with respect to this general technical environment that aspects of the present technology disclosed herein have been contemplated. Furthermore, although a general environment has been discussed, it should be understood that the examples described herein should not be limited to the general environment identified herein.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Among other things, aspects of the present disclosure include systems and methods for calibrating oxygen sensors in ventilators. In an aspect, the technology relates to a system for calibrating an oxygen sensor of one or more ventilators, the system including: an oxygen sensor coupled in flow communication with an oxygen gas supply line, the oxygen gas supply line configured to channel a first flow of oxygen gas with a first unknown oxygen concentration value from a first oxygen gas source; a calibration circuit including a second oxygen gas source coupled in flow communication with the oxygen sensor and a third oxygen gas source coupled in flow communication with the oxygen sensor, wherein the second oxygen gas source is configured to generate a second flow of oxygen gas with a first known oxygen concentration value and the third oxygen gas source is configured to generate a third flow of oxygen gas with a second known oxygen concentration value, the first known oxygen concentration value being different than the second known oxygen concentration value; one or more valves configured to selectively channel either the first flow, the second flow, or the third flow of oxygen gas past the oxygen sensor; and a controller including a processor and memory coupled in communication with the oxygen sensor and the one or more valves, wherein the controller is configured to determine a calibration curve for the oxygen sensor via the calibration circuit by measuring the second flow of oxygen gas with the first known oxygen concentration value and the third flow of oxygen gas with the second known oxygen concentration value and comparing the measured values to the known values, and wherein based on the calibration curve, an oxygen concentration value of the first flow of oxygen gas is measured and the measured oxygen concentration value of the first flow of oxygen gas is distributed to the one or more ventilators.

In an example, the second oxygen source includes a device of 100% oxygen gas. In another example, the third oxygen gas source includes ambient air. In yet another example, the one or more valves include two electromechanical valves coupled in series. In still another example, the oxygen sensor, the calibration circuit, the one or more valves, and the controller are housed within a ventilator, the ventilator coupled in communication with the one or more ventilators. In an example, the oxygen sensor, the calibration circuit, the one or more valves, and the controller are housed within a measurement device, the measurement device coupled in communication with the one or more ventilators. In another example, an outlet is configured discharge the first flow, the second flow, or the third flow of oxygen gas from the oxygen sensor.

In another aspect, the technology relates to a method of calibrating an oxygen sensor in a ventilator, the method including: measuring, at a first oxygen sensor, a first known oxygen concentration of a first flow of oxygen gas; measuring, at the first oxygen sensor, a second known oxygen concentration of a second flow of oxygen gas, wherein the first known oxygen concentration is different than the second known oxygen concentration; generating a calibration curve for the first oxygen sensor based on the measured first known oxygen concentration and the measured second known oxygen concentration; measuring, at the first oxygen sensor, a third unknown oxygen concentration of a third flow of oxygen gas based on the calibration curve; and distributing the measured oxygen concentration of the third flow of oxygen gas to a second oxygen sensor for calibration thereof.

In an example, the first oxygen sensor is disposed in a measurement device and the second oxygen sensor is disposed in a ventilator, the method further includes coupling the measurement device in communication with the ventilator for the distribution of the measured oxygen concentration. In another example, the first oxygen sensor is disposed in a first ventilator and the second oxygen sensor is disposed in a second ventilator, the method further includes coupling the first ventilator in communication with the second ventilator for the distribution of the measured oxygen concentration. In yet another example, the method further includes alternating between the first ventilator and the second ventilator for measuring the third unknown oxygen concentration and distributing the measured oxygen concentration of the third flow of oxygen gas. In still another example, the method further includes calibrating the second oxygen sensor by generating a second calibration curve based on the received measured oxygen concentration and a measurement of ambient air. In an example, measuring the first known oxygen concentration includes emitting the first flow of oxygen gas from a device coupled in flow communication with the first oxygen sensor. In another example, measuring the second oxygen concentration includes channeling the second flow of oxygen gas from ambient air.

In another aspect, the technology relates to a method of calibrating an oxygen sensor in a ventilator coupled in flow communication to an oxygen gas supply line configured to channel a first flow of oxygen gas with an unknown oxygen concentration value, the method including: receiving a measured oxygen concentration value of the first flow of oxygen gas from a device coupled in flow communication to the oxygen gas supply line; measuring, at the oxygen sensor, a first known oxygen concentration of the first flow of oxygen gas; measuring, at the oxygen sensor, a second known oxygen concentration of a second flow of oxygen gas, wherein the first known oxygen concentration is different than the second known oxygen concentration; generating a calibration curve for the oxygen sensor based on the measured first known oxygen concentration and the measured second known oxygen concentration; and storing the calibration curve at the ventilator for operation of a pneumatic system and the oxygen sensor therein.

In an example, measuring the second known oxygen concentration includes channeling the second flow of oxygen gas from ambient air. In another example, receiving the measured oxygen concentration value includes receiving the measured oxygen concentration value from a remote device coupled in flow communication to the oxygen gas supply line. In yet another example, the remote device is another ventilator. In still another example, the remote device is a measurement device. In an example, the method further includes coupling the ventilator in communication with the device.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a schematic diagram illustrating an exemplary ventilator connected to a human patient.

FIG. 2 is a block-diagram illustrating the ventilator shown in FIG. 1.

FIG. 3 is a schematic diagram illustrating a ventilator communication system including the ventilator shown in FIGS. 1 and 2.

FIG. 4 is a schematic view of an exemplary measurement device connected to a gas supply system.

FIG. 5 is a block-diagram illustrating the measurement device shown in FIG. 4.

FIG. 6 is a schematic view of a calibration system for the ventilator or measurement device shown in FIGS. 1-5.

FIG. 7 is a flowchart illustrating a method for calibrating an oxygen sensor in a ventilator.

FIG. 8 is a flowchart illustrating another method for calibrating an oxygen sensor in a ventilator.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. Ventilators include an oxygen sensor so that the respiratory gases have a required or desired oxygen concentration supplied to the patient. This oxygen sensor, however, can have a drift measurement value over time. To address sensor drift, self-calibration of the oxygen sensor periodically occurs between a scheduled maintenance calibration of the entire ventilator. Typically, scheduled maintenance calibration of the ventilator and the oxygen sensor therein occurs every six months to a year on the ventilator. The self-calibration of the oxygen sensor can include using inlet oxygen gas that is supplied to the ventilator as a first calibration point and ambient air for a second calibration point. The inlet oxygen gas is generally a 100% oxygen concentration gas source, and thus, has a known oxygen concentration value. Additionally, ambient air also has a known oxygen concentration value (e.g., 21%) so that a calibration curve for the oxygen sensor is generated based on two points. However, the inlet oxygen gas that is supplied to the ventilator may not be a 100% oxygen concentration gas source.

The systems and methods described herein enable for the oxygen sensor within the ventilator to maintain self-calibration functionality when the inlet oxygen gas to the ventilator has an oxygen concentration level that is not known. For example, the inlet oxygen gas may be a 93% oxygen system which includes oxygen concentration levels between 90% and 100% oxygen. In an example, a calibration circuit having a reliable small source of a known oxygen concentration (e.g., 100% oxygen) and ambient air (e.g., 21% oxygen) is used to calibrate the oxygen sensor by generating a calibration curve. This calibration circuit can be within the ventilator itself or within a remote measurement device coupled to the ventilator. Additionally, once the oxygen sensor is calibrated, the oxygen sensor measures the oxygen concentration value from the inlet oxygen gas and this measured oxygen concentration value can be distributed to a network of other ventilators for self-calibration processes. As such, on demand oxygen sensor calibration can be performed, thereby improving accuracy between calibrations and reducing drift of the oxygen sensors. With these concepts in mind, several exemplary systems and methods are discussed below.

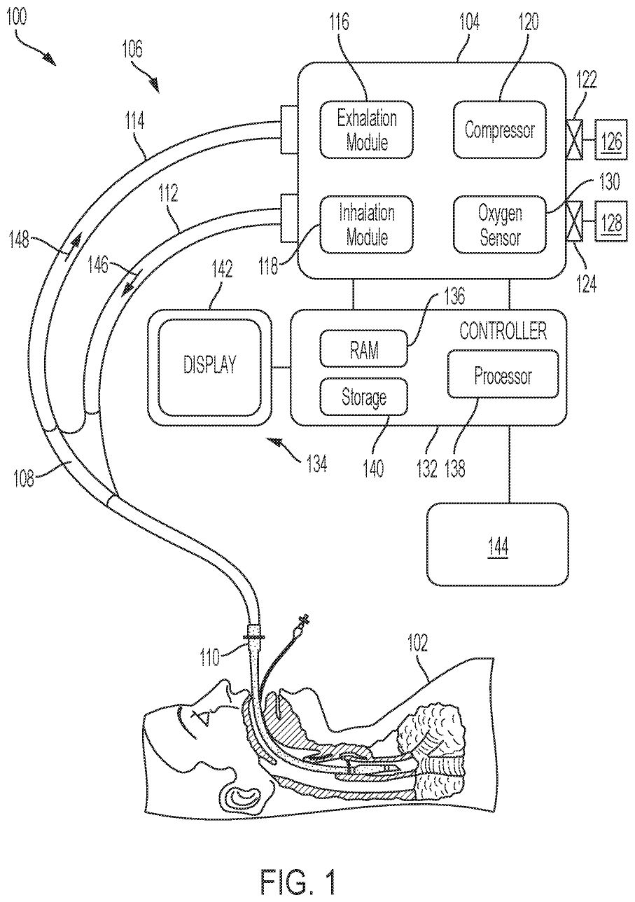

FIG. 1 is a schematic diagram illustrating an exemplary ventilator 100 connected to a human patient 102. The ventilator 100 includes a pneumatic system 104 (also referred to as a pressure generating system 104) for circulating breathing gases to and from the patient 102 via a ventilation tubing system 106 (also referred to as a fluid flow circuit 106), which couples the patient to the pneumatic system 104 via an invasive (e.g., endotracheal tube, as shown) or a non-invasive (e.g., nasal mask) patient interface.

The ventilation tubing system 106 may be a two-limb (shown) or a one-limb circuit for carrying gases to and from the patient 102. In a two-limb example, a fitting, typically referred to as a “wye-fitting” 108, may be provided to couple a patient interface 110 to an inhalation limb 112 and an exhalation limb 114 of the ventilation tubing system 106.

The pneumatic system 104 may have a variety of configurations. In the present example, the system 104 includes an exhalation module 116 coupled with the exhalation limb 114 and an inhalation module 118 coupled with the inhalation limb 112. A compressor 120 or other source(s) of pressurized gases (e.g., air, oxygen, and/or nitrogen) is coupled in flow communication with inhalation module 118 to provide a gas source for ventilatory support via the inhalation limb 112. The pneumatic system 104 may include a variety of other components, including mixing modules, valves, sensors, tubing, accumulators, filters, etc.

In the example, the pneumatic system 104 also includes pressure-regulating valves 122, 124 that control pressurized air and oxygen sources 126, 128, respectively. The pressurized air and oxygen sources 126, 128 may be available from wall outlets or from tanks. These sources are represented as wall outlets in FIG. 1, but may be tanks or other sources as required or desired. The pressure regulating valves (e.g., regulators) 122, 124 control the release of air and oxygen from the sources 126, 128 and each regulating valve regulates flow from its source so that the combined respiratory gas delivered to the patient has a required or desired concentration of oxygen and is supplied to the patient at required or desired pressures and rates. As such, an oxygen sensor 130 is included so as to sample the airflow to the patient and confirm that the appropriate oxygen concentration is being delivered.

A controller 132 is operatively coupled with pneumatic system 104 and an operator interface 134 that may enable a technologist to interact with the ventilator 100 (e.g., change ventilator settings, select operational modes, view monitored parameters, etc.). The controller 132 may include memory 136, one or more processors 138, storage 140, and/or other components of the type found in command and control computing devices. For example, the processor 138 can be a processor, a complex programmable logic device “CPLD,” a field programmable gate array “FPGA,” or a digital signal processor “DSP.” In the example, the operator interface 134 includes a display 142 that may be touch-sensitive and/or voice-activated, enabling the display 142 to serve both as an input and output device.

The memory 136 includes non-transitory, computer-readable storage media that stores software that is executed by the processor 138, and which controls the operation of the ventilator 100. In an example, the memory 136 includes one or more solid-state storage devices such as flash memory chips. In an alternative example, the memory 136 may be mass storage connected to the processor 138 through a mass storage controller (not shown) and a communications bus (not shown). Although the description of computer-readable media contained herein refers to a solid-state storage, it should be appreciated by those skilled in the art that computer-readable storage media can be any available media accessed by the processor 138. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer device. Communication between components of the ventilator 100 or between the ventilator 100 and other therapeutic equipment and/or remote monitoring systems may be conducted via wired or wireless means as required or desired.

The ventilator 100 may engage one or more data collection sensors (e.g., the oxygen sensor 130) to monitor various parameters that may be measured or calculated based on the system between the ventilator 100 and the patient 102. For example, the data collection sensors may collect one or more of gas flow, pressure, volume, gas concentration levels, or any other measurement that may be measured, calculated, or derived based on ventilation of the patient 102, measured at both the inhalation module 118 and exhalation module 116 of the ventilator. While measuring and collecting data, the ventilator 100 may analyze, graph, or perform other calculations to determine other desired parameters, such as an oxygen concentration level at oxygen sensor 130. This measured, collected, or calculated data may be used by the technologist or ventilator 100 when determining potential adjustments or changes to settings of the ventilator 100 in order to optimize patient-ventilator interaction.

In the example, the oxygen sensor 130 can be an electrochemical oxygen sensor or the like that may have a drift measurement value over time. As used herein, “drift” is a slow variation of the accuracy of the oxygen sensor measurements over time and can include variations that result in either a higher measured oxygen concentration value or a lower measured oxygen concentration value. In some known systems, self-calibration of the oxygen sensor 130 is periodically performed between ventilator maintenance calibrations (e.g., 6 months—annually) so as to reduce the drift measurement value of the oxygen sensor. This type of periodic self-calibration of the oxygen sensor 130 can use a first oxygen flow from the wall outlet 128, which has a known oxygen concentration level of about 100% (e.g., between 99%-100% oxygen), an a second oxygen flow from the ambient air, which also has a known oxygen concentration level of about 21%, to generate a calibration curve. This calibration curve is stored within the controller 132 and is used to reduce or prevent drift of the oxygen sensor 130 and between the maintenance calibrations of the system. This calibration method, however, is applicable because the oxygen concentration level of the wall outlet 128 is essentially known and has a value between 99%-100% which is a range that can produce a usable calibration curve for the entire oxygen range for the ventilator 100 during the periods between the maintenance calibrations.

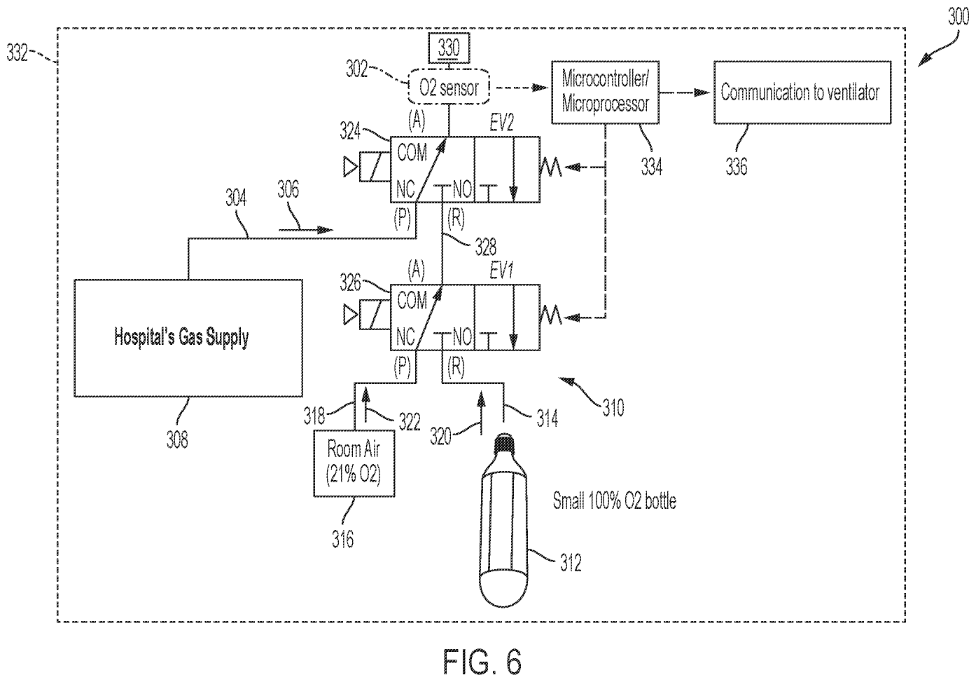

In other systems, however, the oxygen concentration level of the wall outlet 128 is essentially unknown because it is of a reduced concentration level (e.g., between 90%-100% oxygen) and of a range that may not produce a usable calibration curve for the ventilator 100 when using the above described self-calibration process. Accordingly, in the examples described herein a calibration system 144 can be used to determine the oxygen concentration level of the gas being channeled through the wall outlet 128 and supply the oxygen concentration level of the gas to the oxygen sensor of connected ventilators for self-calibration procedures. Additionally, the calibration system 144 can be used with oxygen gas flows of higher concentration values (e.g., 99%-100%) so as to generate a more accurate calibration curve than assuming the oxygen concentration level is about 100%. The calibration system 144 is described further below and in reference to FIG. 6.

In operation of the ventilator 100, the inhalation module 118 is configured to deliver gases (e.g., a flow of fluid) 146 to the patient 102 and the exhalation module 116 is configured to receive gases (e.g., the flow of fluid) 148 from the patient. In aspects, the operation of the ventilator 100 may be based at least partially on the oxygen concentration level of the inhalation flow 146.

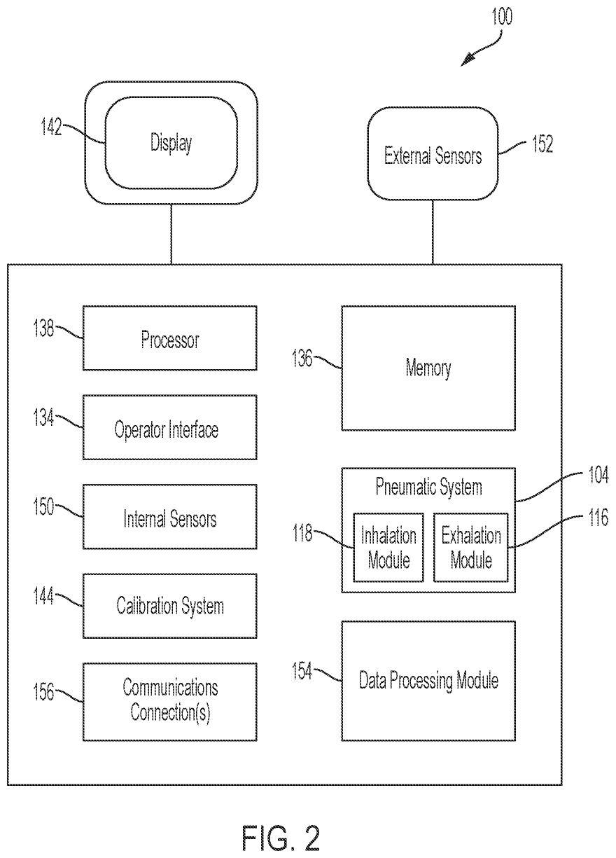

FIG. 2 is a block-diagram illustrating the ventilator 100 with its various operational modules and components. That is, ventilator 100 may include, among other things, the memory 136, one or more processors 138, the operator interface 134, and the pneumatic system 104 (which may further include the inhalation module 118 and the exhalation module 116). The processors 138 may be configured with a clock or other time keeping instrument whereby elapsed time may be monitored by the ventilator 100 and certain operations can be performed at predetermined time sampling periods, intervals, or cycles.

The ventilator 100 may also include the display 142. The display 142 can be integral with the ventilator 100 or a discrete device that is communicatively coupled to ventilator 100. The display 142 provides various input screens, for receiving input from the technologist, and various display screens, for presenting useful information to the technologist. The display 142 is configured to communicate with the operator interface 134 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, alarms, patient information, parameter settings, etc.). The elements may include controls, graphics, charts, tool bars, input fields, icons, etc. Alternatively, other suitable means of communication with the ventilator 100 may be provided, for instance by a wheel, keyboard, mouse, or other suitable interactive device. Thus, the operator interface 134 may accept commands and input through the display 142 as required or desired. The display 142 may also provide information in the form of various ventilatory data regarding the physical condition of a patient and/or a prescribed respiratory treatment. The information may be data that is input into the system, based on data collected by the ventilator 100 and one or more internal 150 or external 152 sensors, derived from data by a data processing module 154, and the useful information may be displayed to the clinician in the form of graphs, wave representations (e.g., a waveform), pie graphs, numbers, or other suitable forms of graphic display. For example, the data processing module 154 may be operative to calibrate oxygen sensors and/or display information regarding the oxygen sensors, as detailed herein.

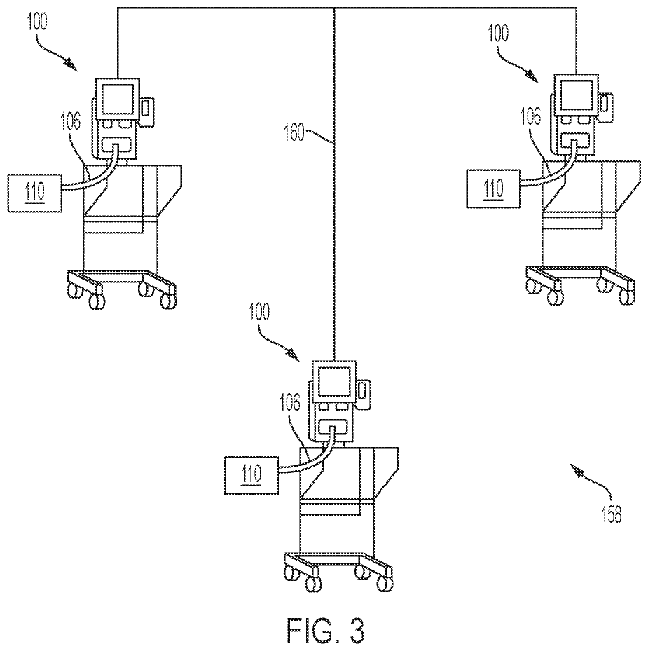

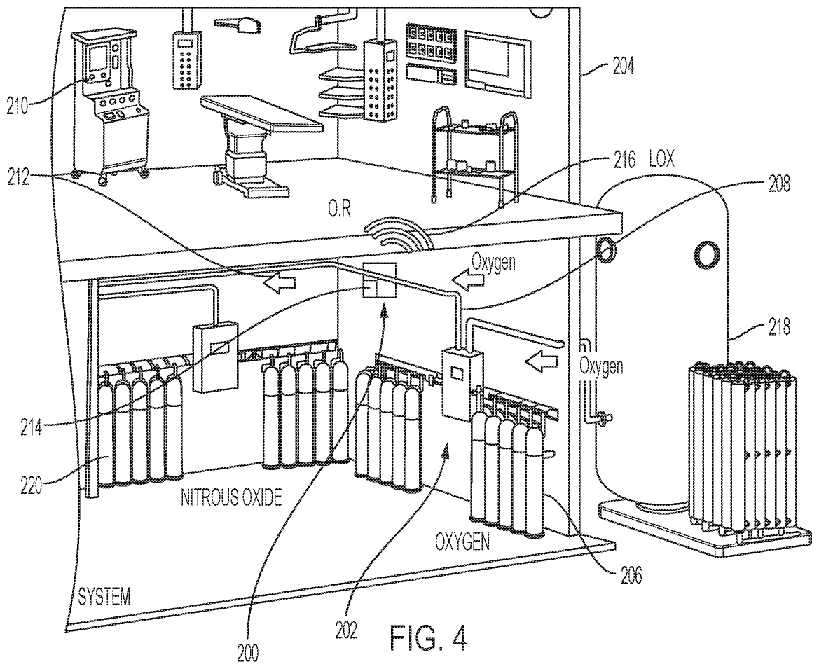

In the example, the ventilator 100 includes communication connections 156 capable of coupling the ventilator 100 in communication with a remote device (not shown) and transmitting data therebetween. For example, transmitting and/or receiving calibration data from the calibration system 144. Examples of remote devices can include, but are not limited to, measurement devices 200 (shown in FIG. 4 below), one or more other ventilators 100 (shown in FIG. 3 below), and the like. The communication connections 156 can be wired (e.g., cables) or wireless (e.g., radio-frequency wireless connections) as required or desired. For example, the communication connections 156 may be a WIFI-based connection, a BLUETOOTH-based connection, an RF-LITE-based connection, a ZIGBEE-based connection, an ultra-wideband-based connection, an Ethernet connection, a network-based connection (e.g., local area network (LAN), wide area network (WAN), etc.), an Internet-based connection, and/or an optical connection, such as an infrared-based connection.

The pneumatic systems 104 may oversee ventilation of a patient according to ventilatory settings. Ventilatory settings may include any appropriate input for configuring the ventilator to deliver breathable gases to a particular patient, including measurements and settings associated with oxygen concentration of the breathing circuit. Ventilatory settings may be entered by a technologist, e.g., based on a prescribed or target treatment protocol for the particular patient, or automatically generated by the ventilator, e.g., based on attributes (i.e., age, diagnosis, ideal body weight, gender, etc.) of the particular patient according to any appropriate standard protocol or otherwise. In some cases, certain ventilatory settings may be adjusted based on the oxygen concentration level, e.g., to optimize the prescribed treatment. Ventilatory settings may include inhalation flow rate and/or pressure, frequency of delivered breaths (e.g., respiratory rate), tidal volume, positive end-expiratory pressure “PEEP,” etc.

FIG. 3 is a schematic diagram illustrating a ventilator communication system 158 including the ventilators 100 described above in reference to FIGS. 1 and 2. Certain components are described above, and thus, are not necessarily described further. In the system 158, a plurality of ventilators 100 are coupled in communication 160 to one another. This coupling can be wired or wireless as described herein and as required or desired. In the example, one or more of the ventilators 100 includes the calibration system 144 (shown in FIGS. 1 and 2) that measures the oxygen concentration level of the oxygen gas flow from the wall outlet and distributes this measured oxygen concentration level to the other ventilators 100. As such, a first ventilator can measure the oxygen concentration level of the gas flow from the wall outlet and send the measured concentration level to a second different ventilator. The second ventilator then can use the measured values for self-calibration of its oxygen sensor 130 (shown in FIG. 1).

In an aspect, each of the ventilators 100 in the system 158 can include the calibration system 144, and each of the ventilators 100 can alternate measuring the oxygen concentration level of the oxygen gas flow and distributing this measurement to the others. By alternating the measurement of oxygen concentration level between the ventilators 100, sensor drift of each of the calibration systems 144 is reduced thereby increasing accuracy. This alternating between ventilators can be sequence based, or based on use, non-use, location, or any other contention of the ventilator as required or desired. In another example, a subset of the plurality of ventilators 100 (e.g., 25%, 40%, 50%, 75%, etc.) include the calibration systems 144 for measuring the oxygen concentration levels and distributing this measurement to others. By having only a subset of ventilators 100 have the calibration system 144, the cost efficiency of the system 158 is increased.

FIG. 4 is a schematic view of an exemplary measurement device 200 connected to a gas supply system 202. The gas supply system 202 is located within a structure 204 (e.g., a hospital or the like) and includes oxygen sources 206 coupled to one or more oxygen supply lines 208. One or more ventilators 210 are coupled in flow communication with the oxygen supply line 208 and are configured to receive a flow of oxygen gas 212 from the source 206. The ventilators 210 can be located throughout the structure 204, and for example, on different floors of the structure 204. In the example, the oxygen gas 212 may be oxygen 93 system with an oxygen concentration between 90% and 100%. In another example, the oxygen gas 212 may have an oxygen concentration between 99% and 100%. In an aspect, the oxygen gas 212 may correspond to the ISO standard 80601-2-1.

The measurement device 200 includes a calibration system 214 that can be used to measure the oxygen concentration level of the gas 212 being channeled through the supply line 208 and supply the measured oxygen concentration level of the gas to the ventilator 210 for self-calibration procedures. In examples, the measurement device 200 is coupled in communication 216 with the ventilators 210 so that oxygen concentration measurements can be sent from the measurement device 200 and received by the ventilator 210. This communication 216 can be via wired or wireless (as illustrated) and as required or desired. The calibration system 214 is described further below and in reference to FIG. 6. In examples, the gas supply system 202 for structure 204 may include a liquid oxygen source 218 and or a nitrous oxide source 220 as required or desired. In contrast to the ventilator only system shown in FIG. 3, utilization of the measurement device 200 enables a single device to measure oxygen concentration levels and distribute the measurement data to one or more of the ventilators 100 coupled thereto. This configuration increases cost efficiencies for the ventilators 100 and also enables an increased accuracy calibration system 214.

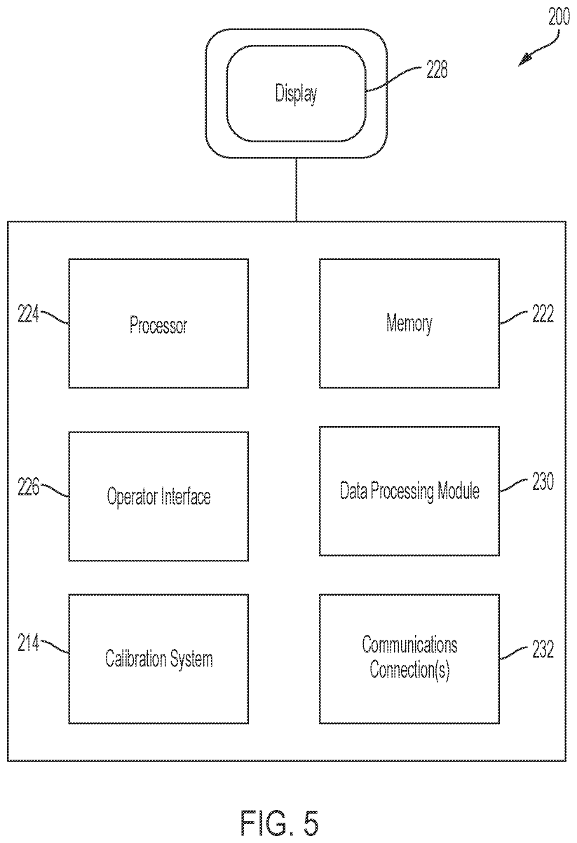

FIG. 5 is a block-diagram illustrating the measurement device 200 with its various operational modules and components. That is, the measurement device 200 may include, among other things, memory 222, one or more processors 224, an operator interface 226, and the calibration system 214. The processors 224 may be configured with a clock or other time keeping instrument whereby elapsed time may be monitored by the measurement device 200 and certain operations can be performed at predetermined time sampling periods, intervals, or cycles.

The measurement device 200 may also include a display 228. The display 228 can be integral with the measurement device 200 or a discrete device that is communicatively coupled to measurement device 200. The display 228 provides various input screens, for receiving input from the operator, and various display screens, for presenting useful information to the operator. The display 228 is configured to communicate with the operator interface 226 and may include a graphical user interface (GUI). The GUI may be an interactive display, e.g., a touch-sensitive screen or otherwise, and may provide various windows (i.e., visual areas) comprising elements for receiving user input and interface command operations and for displaying oxygen sensor information (e.g., data, alerts, alarms, oxygen level information, parameter settings, etc.). The elements may include controls, graphics, charts, tool bars, input fields, icons, etc. Alternatively, other suitable means of communication with the measurement device may be provided, for instance by a wheel, keyboard, mouse, or other suitable interactive device. Thus, the operator interface 226 may accept commands and input through the display 228 as required or desired. The display 228 may also provide information in the form of various oxygen sensor data. The information may be data that is input into the system, based on data collected by the measurement device 200 and the calibration system 214, derived from data by a data processing module 230, and the useful information may be displayed as required or desired. For example, the data processing module 230 may be operative to calibrate oxygen sensors and/or display information regarding the oxygen sensors, as detailed herein.

In the example, the measurement device 200 also includes communication connections 232 capable of coupling the measurement device 200 in communication with a remote device (e.g., the ventilator 210 shown in FIG. 4) and transmitting data therebetween. For example, transmitting and/or receiving calibration data from the calibration system 214. The communication connections 232 can be wired (e.g., cables) or wireless (e.g., radio-frequency wireless connections) as required or desired. For example, the communication connections 232 may be a WIFI-based connection, a BLUETOOTH-based connection, an RF-LITE-based connection, a ZIGBEE-based connection, an ultra-wideband-based connection, an Ethernet connection, a network-based connection (e.g., local area network (LAN), wide area network (WAN), etc.), an Internet-based connection, and/or an optical connection, such as an infrared-based connection.

FIG. 6 is a schematic view of a calibration system 300 for the ventilator 100 (e.g., calibration system 144 shown in FIGS. 1 and 2) or the measurement device 200 (e.g., calibration system 214 shown in FIGS. 4 and 5). The calibration system 300 is configured to measure oxygen concentration level of an oxygen gas flow for use with the calibration of an oxygen sensor of one or more ventilators that are attached thereto. As described herein, the calibration system 300 can be integrated within one or more ventilators (e.g., ventilator 100) so that a network of ventilators (e.g., as shown in FIG. 3) is created for calibrating the oxygen sensors thereof, or can be housed within a measurement device (e.g., measurement device 200) so that a dedicated device is utilized for calibrating the oxygen sensors in each of the ventilators (e.g., as shown in FIG. 4).

In the example, the calibration system 300 includes an oxygen sensor 302. In an aspect, the oxygen sensor 302 may be the oxygen sensor 130 (shown in FIG. 1) of the ventilator 100. In another example, the oxygen sensor 302 may be discrete and separate from the oxygen sensor 130 of the ventilators but still disposed within the ventilator 100. In yet another example, the oxygen sensor 302 may be within the measurement device 200. The oxygen sensor 302 is coupled in flow communication with a first oxygen gas supply line 304 that is configured to channel a first flow of oxygen gas 306 with an unknown oxygen concentration value from a first oxygen gas source 308. In an aspect, the unknown oxygen concentration value of the oxygen gas 306 is between 90% and 100%. This oxygen gas 306 is also the gas that is provided to the ventilators for operation thereof. In an example, the oxygen sensor 302 can be an electrochemical oxygen sensor or the like, and as such, a sensor that is the same or similar to the oxygen sensors of the ventilators. With this type of sensor, sensor drift needs to be accounted for and the sensor calibrated. In other examples, the oxygen sensor 302 can include paramagnetic oxygen sensor, zirconium, or the like. With these types of sensors, the sensor may only be required to be calibrated once and/or they achieve very high accuracy when compared to the electrochemical sensor, however, system costs will be increased.

The calibration system 300 also includes a calibration circuit 310 that is configured to calibrate the calibration oxygen sensor 302. The calibration circuit 310 includes a second oxygen gas source 312 coupled in flow communication with the oxygen sensor 302 via a second gas supply line 314 and a third oxygen gas source 316 coupled in flow communication with the oxygen sensor 302 via a third gas supply line 318. The second oxygen gas source 312 is configured to generate a second flow of oxygen gas 320 towards the oxygen sensor 302 and the third oxygen gas source 316 is configured to generate a third flow of oxygen gas 322 towards the oxygen sensor 302. The second flow of oxygen gas 320 has a first known oxygen concentration value. In an aspect, the first known oxygen concentration value is 100% oxygen gas and the gas source 312 is a bottle of oxygen gas so that the calibration circuit 310 can be self-contained within the calibration system 300. By using a bottle of oxygen gas, the gas source 312 is replaceable/rechargeable as required or desired. It should be appreciated that the bottle of oxygen gas can be any other device, such as a bottle, box, recipient, or any other rechargeable/disposable device with a constant and known oxygen level.

The third flow of oxygen gas 322 has a second known oxygen concentration value. In an aspect, the second known oxygen concentration value is 21% oxygen ambient air and the space around the system 300 is used as the gas source 316. The first known oxygen concentration value is different than the second known oxygen concentration value so that a calibration curve can be generated based on two points. By having the known oxygen concentration values correspond to the upper and lower limits of oxygen concentration values, the calibration curve that is generated can be more accurate. It should be appreciated, however, that other values of known oxygen concentration sources other than those described herein can be used as required or desired as long as the values are constant and known. For example, oxygen concentration levels of 98% or 99% can be used as required or desired, although using a 100% oxygen source will be free of other gases that may slightly effect the calibration and the accuracy as described herein.

Furthermore, apart from two point calibrations, it is also possible to perform a one point calibration curve at either the first known oxygen concentration value (e.g., a 100% oxygen source) or the second known oxygen concentration value (e.g., the 21% ambient air) while assuming a linear slope of the calibration curve. Using a one point calibration curve, however, may decrease calibration accuracy in the oxygen concentration levels that are further away from the calibration point when compared to using a two point calibration curve.

Two electromechanical valves 324, 326 are configured to couple to the supply lines 304, 314, 318 and selectively channel either the first flow 306, the second flow 320, or the third flow 322 of oxygen gas through the oxygen sensor 302. In the example, the valves 324, 326 are coupled in series to one another with a fourth supply line 328. In the example, the valves 324, 326 are electromagnetically controlled valves (e.g., a solenoid valves) so that valve position can be adjusted via electric current channeled to a valve coil, and thereby regulating fluid flow therethrough. It should be appreciated, that the valves 324, 326 may be any other type of flow control valve, (e.g., pneumatic, motor, etc.) that enables the calibration system 300 to function as described herein.

An outlet 330 is positioned downstream of the oxygen sensor 302 and is configured to discharge the flows 306, 320, 322 from a housing 332 that the calibration system 300 is disposed within. While one example of a fluid flow circuit upstream of the oxygen sensor 302 is illustrated in FIG. 4, it should be appreciated that other configurations are also contemplated here that enable the calibration system 300 to function as described herein.

The calibration system 300 also includes a controller 334 configured to determine a calibration curve for the calibration oxygen sensor 302 based on the calibration circuit 310. The controller 334 includes a process and memory, and is coupled in communication with the oxygen sensor 302 and the valves 324, 326. In operation, the calibration curve is determined by measuring the second flow of oxygen gas 320 with the first known oxygen concentration value (e.g., 100% oxygen) and measuring the third flow of oxygen gas 322 with the second known oxygen concentration value (e.g., 21% ambient air). These measured values are then compared to the known values so as to determine the calibration curve and account for drift of the oxygen sensor 302. Based on the calibration curve, the first flow of oxygen gas 306 is measured for oxygen concentration value and the measured oxygen concentration value is distributed to the oxygen sensor of the ventilators. In the example, the calibration system 300 can use a communication connector 336 for distributing the measured oxygen concentrations. The communication connector 336 can be wired or wireless (e.g., LAN, WAN, point-to-point, Bluetooth, RF, etc.) to communicate with other remote devices.

In examples, the calibration system 300 can be housed within a ventilator (e.g., the ventilator 100) and coupled to the control system for the oxygen sensor 130. Accordingly, the calibration circuit 310 can be used for self-calibration of the oxygen sensor 130 as described herein. In one aspect, the calibration system 300 may be integrated within the ventilator 100 such that the oxygen sensor 302 and the oxygen sensor 130 are the same sensor. In other aspects, the calibration system 300 may be an accessory coupled to the ventilator 100 and the system has both of the two sensors 302 and 130. The calibration sensor 302 measures the oxygen concentration from the wall air for calibrating the ventilator sensor 130.

Additionally, when the calibration system is housed within a ventilator, the ventilator can be coupled in communication with other remote ventilators. This configuration enables multiple ventilators to be connected in a network and receive the measured oxygen concentrations from the calibration system 300. In an aspect, each of these connected ventilators can alternate measurement determinations so as to reduce drift across the calibration oxygen sensors 302. In this example, the measured oxygen concentration level of the gas flow 306 is used by the each of the remote ventilators for self-calibration of the internal oxygen sensors. For example, using the known oxygen concentration level of the gas flow 306 and a known oxygen concentration level of the ambient air to generate a calibration curve for the oxygen sensor 130 for use during operation of the remote ventilator.

In other examples, the calibration system 300 can be housed within a measurement device (e.g., the measurement device 200). This configuration enables a single device to be connected to multiple ventilators and distribute the measured oxygen concentrations to each of the ventilators. As such, each of the ventilators need not include the calibration system 300. In this example, onetime calibration oxygen sensors (e.g., paramagnetic, zirconium, and the like) can be used and so that the calibration circuit 310 can be eliminated as these sensors may not have drift like the others. While onetime calibration sensors are more expensive, by using a dedicated measurement device only one oxygen sensor is needed to measure the oxygen concentration of the oxygen source 308 and distribute this measurement to multiple ventilators connected thereto.

In some examples, the controller 334 of the calibration system 300 may be part of the ventilator controller 132 (shown in FIG. 1) or the measurement device controller (shown in FIG. 5) and the oxygen sensors in the system may be controlled by the same components. In other examples, controllers for the calibration system 300 and the ventilator/measurement device may be discrete components that are coupled in communication together.

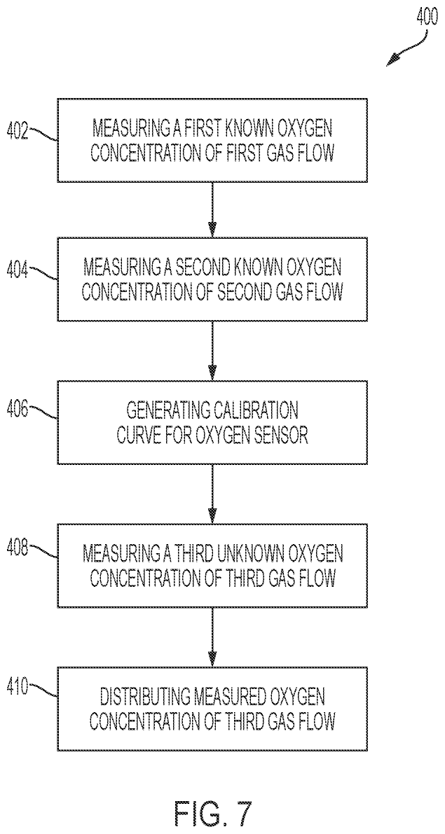

FIG. 7 is a flowchart illustrating a method 400 for calibrating an oxygen sensor in a ventilator. The example methods and operations can be implemented or performed by the systems and devices described herein (e.g., ventilator 100, measurement device 200). The method 400 begins at a first oxygen sensor with measuring a first known oxygen concentration of a first flow of oxygen gas (operation 402). Additionally, at the first oxygen sensor a second known oxygen concentration of a second flow of oxygen gas is measured (operation 404). The first known oxygen concentration is different than the second known oxygen concentration such that a calibration curve for the first oxygen sensor is generated (operation 406) based on the measured first known oxygen concentration and the measured second known oxygen concentration.

In the example, measuring the first known oxygen concentration of the first flow can include emitting the first flow oxygen gas from a first oxygen source coupled in flow communication with the first oxygen sensor. In an aspect, the first oxygen source can be a discrete device of 100% oxygen (e.g., bottle, box, recipient or any other rechargeable/disposable oxygen source) and a predetermined volume charge can be channeled towards the first oxygen sensor for measurement. Additionally, measuring the second known oxygen concentration of the second flow can include channeling the second flow of oxygen gas from a second oxygen source coupled in flow communication with the first oxygen sensor. In an aspect, the second oxygen source can be ambient air (e.g., about 21% oxygen). The first oxygen sensor, and the first and second oxygen sources are part of a calibration circuit that can selectively (e.g., periodically) be used to generate the calibration curve for the first oxygen sensor. Generating the calibration curve includes comparing the measured concentration values for each of the first and second flows to the stored known concentration values and then extrapolating calibration data for concentration levels therebetween. In an aspect, the first and second oxygen source correspond to oxygen concentration levels that correspond to a high concentration level (e.g., 100% oxygen) and a low concentration level (e.g., 21% oxygen) so that extrapolating calibration data is more accurate. It should be appreciated that using other known oxygen concentration levels (e.g., 95% and 30% or the like) to generate the calibration curve for the first oxygen sensor is also contemplated herein. Additionally, by calibrating the first oxygen sensor before measuring unknown oxygen concentration levels, any drift that occurs in the first oxygen sensor can be accounted for and reduced or eliminated.

Turning back to the method 400, once the calibration curve is generated, the method 400 can further include measuring at the first oxygen sensor a third unknown oxygen concentration of a third flow of oxygen gas (operation 408) based on the calibration curve. In the example, measuring the third unknown oxygen concentration of the third flow can include extracting or sampling the third flow of oxygen gas from a third fluid source that is coupled in flow communication with the first oxygen sensor. In an aspect, the third fluid source is a wall outlet of the hospital or other building structure. In an example, the third fluid source is an oxygen 93 system source (e.g., 90%-100% oxygen concentration). In other examples, the third fluid source can have an oxygen concentration of between 99%-100%.

The measured oxygen concentration of the third flow of oxygen gas at the first oxygen sensor is then distributed to a second oxygen sensor for use in calibration thereof (operation 410). The second oxygen sensor also being coupled in flow communication with the third fluid source. To calibrate the second oxygen sensor, the third flow of oxygen gas from the third fluid source now has a known oxygen concentration value, from the received measurement by the first oxygen sensor, and thereby, can be used with another known oxygen concentration value to generate a second calibration curve for the second oxygen sensor and self-calibration thereof. In an example, this other known oxygen concentration value can be from the ambient air (e.g., around 21%). By using the first oxygen sensor to help calibrate the second oxygen sensor, the accuracy of the second calibration is increased. Additionally, the oxygen sensors do not need to be attached to an oxygen source of known concretion at the wall outlet and hospital supply.

In an example, the first oxygen sensor can be disposed in a measurement device and the second oxygen sensor is disposed in a ventilator. One aspect of this configuration is illustrated in FIG. 4 described above. As such, the method 400 can further include coupling the measurement device in communication with the ventilator for the distribution of the measured oxygen concentration. This coupling can be wired or wireless as required or desired. In other examples, the first oxygen sensor is disposed in a first ventilator and the second oxygen sensor is disposed in a second ventilator. One aspect of this configuration is illustrated in FIG. 3 described above. As such, the method can further include coupling the first ventilator in communication with the second ventilator for the distribution of the measured oxygen concentration. When two or more ventilators are used for oxygen sensor calibration, the ventilator that measures and distributes the oxygen concertation of the third flow of oxygen gas can alternate between the first and second ventilators.

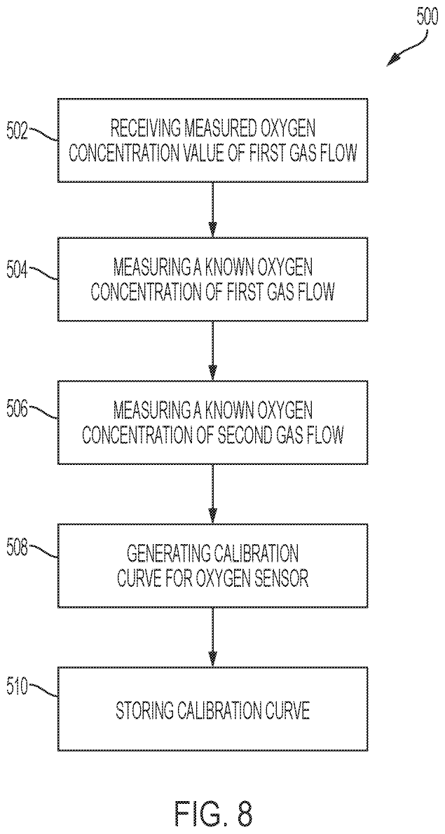

FIG. 8 is a flowchart illustrating another method 500 for calibrating an oxygen sensor in a ventilator. The example methods and operations can be implemented or performed by the systems and devices described herein (e.g., ventilator 100). In some known systems, the ventilator can self-calibrate its oxygen sensor when the supply line oxygen gas is of a known concentration. In an aspect, this is typically between 99%-100%. However, when the supply line oxygen gas is of unknown concentration, or a more accurate calibration curve is required or desired, then the calibration system as described herein is utilized. The method 500 described below relates to the operation of each of the individual ventilators once receiving the measured oxygen calibration levels from the calibration system.

The ventilator in method 500 can be coupled in flow communication to an oxygen gas supply line configured to channel a first flow of oxygen gas with an unknown oxygen concentration value. In an aspect, the oxygen gas supply line is a wall outlet of the hospital or other building structure. In an example, the oxygen gas supply line is an oxygen 93 system source (e.g., 90%-100% oxygen concentration). In other examples, the oxygen gas supply line can have an oxygen concentration of between 99%-100%. The method 500 begins with receiving a measured oxygen concentration value of the first flow of oxygen gas (operation 502) from a device also coupled in flow communication to the oxygen gas supply line. In an example, this device can be a remote device from the ventilator, and for example, another ventilator like the configuration shown in FIG. 3 described above, or a measurement device like the configuration shown in FIG. 4 described above. As such, the ventilator can be coupled in communication with the device in aspects of the method 500. In another example, this device can be internal with the ventilator and when the ventilator itself includes the calibration system as described herein.

Once the oxygen concentration of the oxygen gas supply line is known, the method 500 includes measuring at the oxygen sensor a first known oxygen concentration of the first flow of oxygen gas (operation 504). Additionally, at the oxygen sensor a second known oxygen concentration of a second flow of oxygen gas is measured (operation 506). In the example, measuring the first known oxygen concentration of the first flow can include extracting or sampling the first flow of oxygen gas from a first fluid source that is coupled in flow communication with the oxygen sensor. In an aspect, the first fluid source is a wall outlet of the hospital or other building structure. In an example, the first fluid source is an oxygen 93 system source (e.g., 90%-100% oxygen concentration). In other examples, the first fluid source can have an oxygen concentration of between 99%-100%. Measuring the second known oxygen concentration of the second flow can include channeling the second flow of oxygen gas from a second oxygen source coupled in flow communication with the oxygen sensor. In an aspect, the second oxygen source can be ambient air (e.g., about 21% oxygen).

The measured oxygen concentration that is received at the ventilator of the first flow is different than the known oxygen concentration of the second flow that is measured at the ventilator such that a calibration curve for the oxygen sensor is generated (operation 508) based on the measurement of the first flow and second flow of oxygen gas. Generating the calibration curve includes comparing the measured concentration values for each of the first and second flows to the stored known concentration values and then extrapolating calibration data for concentration levels therebetween. In an aspect, the first and second oxygen source correspond to oxygen concentration levels that correspond to a high concentration level (e.g., 90%-100% oxygen) and a low concentration level (e.g., 21% oxygen) so that extrapolating calibration data is more accurate. It should be appreciated that using other known oxygen concentration levels to generate the calibration curve for the oxygen sensor is also contemplated herein. The calibration curve can then be stored at the ventilator for operation of a pneumatic system and the oxygen sensor therein (operation 510). By calibrating the oxygen sensor before operation of the pneumatic system, any drift that occurs in the oxygen sensor can be accounted for and reduced or eliminated.

In the examples, electronic hardware and valves are used to calibrate an oxygen sensor in the proposed device so that it can accurately measure the oxygen concentration level in the incoming gas supply. This accurate measurement of the gas supply oxygen concentration can be distributed to ventilators and be used as a known oxygen concentration value for calibration of the oxygen sensor of the ventilator. The distribution of the measured oxygen concentration from the gas supply to the ventilators can be performed via wireless or wired connections. With this system, on demand oxygen sensor calibration can be performed, thereby improving accuracy between calibrations and reducing drift of the oxygen sensors.

Although the present disclosure discusses the implementation of these techniques in the context of a ventilator capable of calibrating oxygen sensors, the techniques introduced above may be implemented for a variety of medical devices or devices utilizing sensors and control valves. A person of skill in the art will understand that the technology described in the context of a medical ventilator for human patients could be adapted for use with other systems such as ventilators for non-human patients, or general gas transport systems. Additionally, a person of ordinary skill in the art will understand that calibrating oxygen sensors may be implemented in a variety of breathing circuit setups that may have a sensor and control valves. Further, while described primarily for use with an oxygen sensor, the techniques described herein may also be used at other sensors of the ventilator as required or desired.

Those skilled in the art will recognize that the methods and systems of the present disclosure may be implemented in many manners, and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about,” “approximately,” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure.

What is claimed is:

1. A system for calibrating an oxygen sensor of one or more ventilators, the system comprising:
an oxygen sensor coupled in flow communication with an oxygen gas supply line, the oxygen gas supply line configured to channel a first flow of oxygen gas with a first unknown oxygen concentration value from a first oxygen gas source;
a calibration circuit including a second oxygen gas source coupled in flow communication with the oxygen sensor and a third oxygen gas source coupled in flow communication with the oxygen sensor, wherein the second oxygen gas source is configured to generate a second flow of oxygen gas with a first known oxygen concentration value and the third oxygen gas source is configured to generate a third flow of oxygen gas with a second known oxygen concentration value, the first known oxygen concentration value being different than the second known oxygen concentration value;
one or more valves configured to selectively channel either the first flow, the second flow, or the third flow of oxygen gas past the oxygen sensor; and
a controller comprising a processor and memory coupled in communication with the oxygen sensor and the one or more valves, wherein the controller is configured to determine a calibration curve for the oxygen sensor via the calibration circuit by measuring the second flow of oxygen gas with the first known oxygen concentration value and the third flow of oxygen gas with the second known oxygen concentration value and comparing the measured values to the known values, and wherein based on the calibration curve, an oxygen concentration value of the first flow of oxygen gas is measured and the measured oxygen concentration value of the first flow of oxygen gas is distributed to the one or more ventilators.

2. The system of claim 1, wherein the second oxygen source includes a device of 100% oxygen gas.

3. The system of claim 1, wherein the third oxygen gas source includes ambient air.

4. The system of claim 1, wherein the one or more valves include two electromechanical valves coupled in series.

5. The system of claim 1, wherein the oxygen sensor, the calibration circuit, the one or more valves, and the controller are housed within a ventilator, the ventilator coupled in communication with the one or more ventilators.

6. The system of claim 1, wherein the oxygen sensor, the calibration circuit, the one or more valves, and the controller are housed within a measurement device, the measurement device coupled in communication with the one or more ventilators.

7. The system of claim 1, further comprising an outlet configured discharge the first flow, the second flow, or the third flow of oxygen gas from the oxygen sensor.

8. A method of calibrating an oxygen sensor in a ventilator, the method comprising:
measuring, at a first oxygen sensor, a first known oxygen concentration of a first flow of oxygen gas;
measuring, at the first oxygen sensor, a second known oxygen concentration of a second flow of oxygen gas, wherein the first known oxygen concentration is different than the second known oxygen concentration;
generating a calibration curve for the first oxygen sensor based on the measured first known oxygen concentration and the measured second known oxygen concentration;
measuring, at the first oxygen sensor, a third unknown oxygen concentration of a third flow of oxygen gas based on the calibration curve; and
distributing the measured oxygen concentration of the third flow of oxygen gas to a second oxygen sensor for calibration thereof.

9. The method of claim 8, wherein the first oxygen sensor is disposed in a measurement device and the second oxygen sensor is disposed in a ventilator, the method further comprising coupling the measurement device in communication with the ventilator for the distribution of the measured oxygen concentration.

10. The method of claim 8, wherein the first oxygen sensor is disposed in a first ventilator and the second oxygen sensor is disposed in a second ventilator, the method further comprising coupling the first ventilator in communication with the second ventilator for the distribution of the measured oxygen concentration.

11. The method of claim 10, further comprising alternating between the first ventilator and the second ventilator for measuring the third unknown oxygen concentration and distributing the measured oxygen concentration of the third flow of oxygen gas.

12. The method of claim 8, further comprising calibrating the second oxygen sensor by generating a second calibration curve based on the received measured oxygen concentration and a measurement of ambient air.

13. The method of claim 8, wherein measuring the first known oxygen concentration includes emitting the first flow of oxygen gas from a device coupled in flow communication with the first oxygen sensor.

14. The method of claim 8, wherein measuring the second oxygen concentration includes channeling the second flow of oxygen gas from ambient air.

15. A method of calibrating an oxygen sensor in a ventilator coupled in flow communication to an oxygen gas supply line configured to channel a first flow of oxygen gas with an unknown oxygen concentration value, the method comprising:
receiving a measured oxygen concentration value of the first flow of oxygen gas from a device coupled in flow communication to the oxygen gas supply line;
measuring, at the oxygen sensor, a first known oxygen concentration of the first flow of oxygen gas;
measuring, at the oxygen sensor, a second known oxygen concentration of a second flow of oxygen gas, wherein the first known oxygen concentration is different than the second known oxygen concentration;
generating a calibration curve for the oxygen sensor based on the measured first known oxygen concentration and the measured second known oxygen concentration; and
storing the calibration curve at the ventilator for operation of a pneumatic system and the oxygen sensor therein.

16. The method of claim 15, wherein measuring the second known oxygen concentration includes channeling the second flow of oxygen gas from ambient air.

17. The method of claim 15, wherein receiving the measured oxygen concentration value includes receiving the measured oxygen concentration value from a remote device coupled in flow communication to the oxygen gas supply line.

18. The method of claim 17, wherein the remote device is another ventilator.

19. The method of claim 17, wherein the remote device is a measurement device.

20. The method of claim 15, further comprising coupling the ventilator in communication with the device.
